(19) United States
(12) Patent Application Publication
(10) Pub. No.: US 2007/0196844 A1
(43) Pub. Date: Aug. 23, 2007

(54) Protein PDX1 as a marker for breast cancer

(76) Inventors: Gabriele Pestlin, Muenchen (DE); Herbert Andres, Penzberg (DE); Marie-Luise Hagmann, Penzberg (DE); Johann Karl, Peissenberg (DE); Michael Thierolf, Penzberg (DE); Werner Zolg, Weilheim-Unterhausen (DE); Peter Berndt, Basel (CH); Hanno Langen, Loerrach (DE)

Correspondence Address: ROCHE DIAGNOSTICS OPERATIONS INC., 9115 Hague Road, Indianapolis, IN, 46250-0457, US

(21) Appl. No.: 11/640,523

(22) Filed: Dec. 14, 2006

Related U.S. Application Data
(63) Continuation of application No. PCT/EP05/06528, filed on Jun. 17, 2005.

(30) Foreign Application Priority Data
Jun. 18, 2004 (EP) 04014315.8

(51) Int. Cl. C12Q 1/68; G01N 33/574; C12M 3/00

(52) U.S. Cl. 435/6; 435/7.23; 435/287.2

(57) ABSTRACT

The present invention relates to the diagnosis of breast cancer. It discloses the use of protein PDX1 (peroxiredoxin 1) in the diagnosis of breast cancer. It relates to a method for diagnosis of breast cancer from a liquid sample, derived from an individual by measuring PDX1 in said sample. Measurement of PDX1 can, e.g., be used in the early detection or diagnosis of breast cancer.

### RELATED APPLICATIONS

This application is a continuation of PCT/EP2005/006528 filed Jun. 17, 2005 and claims priority to EP EP 04014315.8 filed Jun. 18, 2004.

### FIELD OF THE INVENTION

The present invention relates to the diagnosis of breast cancer. It discloses the use of PDX1 (peroxiredoxin 1) in the diagnosis of breast cancer. Furthermore, it especially relates to a method for diagnosis of breast cancer from a liquid sample, derived from an individual by measuring PDX1 in said sample. Measurement of PDX1 can, e.g., be used in the early detection or diagnosis of breast cancer or in the surveillance of patients who undergo surgery.

### BACKGROUND OF THE INVENTION

Cancer remains a major public health challenge despite progress in detection and therapy. Amongst the various types of cancer, breast cancer (BC) is one of the most frequent cancers among women in the Western world.

The staging of cancer is the classification of the disease in terms of extent, progression, and severity. It groups cancer patients so that generalizations can be made about prognosis and the choice of therapy.

Today, the TNM system is the most widely used classification of the anatomical extent of cancer. It represents an internationally accepted, uniform staging system. There are three basic variables: T (the extent of the primary tumor), N (the status of regional lymph nodes) and M (the presence or absence of distant metastases). The TNM criteria are published by the UICC (International Union Against Cancer) (Sobin, L. H., Wittekind, Ch. (eds): TNM Classification of Malignant Tumours, fifth edition, 1997). The staging system for breast cancer has recently been revised (Singletary, S. E., et al., J. Clin. Oncol. 20 (2002) 3628-3636).

What is especially important is that early diagnosis of BC translates to a much better prognosis. Therefore, best prognosis have those patients as early as in stage Tᵢₛ, N0, M0 or T1-3; N0; M0, if treated properly have a more than 90% chance of survival 5 years after diagnosis as compared to a 5-years survival rate of only 18% for patients diagnosed when distant metastases are already present.

In the sense of the present invention early diagnosis of BC refers to a diagnosis at a pre-cancerous state, ductal carcinoma in situ (DCIS) or at a tumor stage where no metastases at all (neither proximal nor distal), i.e., Tᵢₛ, N0, M0 or T1-4; N0; M0 are present. Tᵢₛ denotes carcinoma in situ.

In a preferred embodiment the detection of PDX1 is used to diagnose BC in a non-metastatic stage, i.e., that diagnosis is made at stage Tᵢₛ, N0, M0 or T1-3; N0; M0 (Tᵢₛ-3; N0; M0).

The earlier cancer can be detected/diagnosed, the better is the overall survival rate. This is especially true for BC. The prognosis in advanced stages of tumor is poor. More than one third of the patients will die from progressive disease within five years after diagnosis, corresponding to a survival rate of about 40% for five years. Current treatment is only curing a fraction of the patients and clearly has the best effect on those patients diagnosed in an early stage of disease.

With regard to BC as a public health problem, it is essential that more effective screening and preventative measures for breast cancer will be developed.

The earliest detection procedures available at present for breast cancer involve using clinical breast examination and mammography. However, significant tumor size must typically exist before a tumor is palpable or can be detected by a mammogram. The density of the breast tissue and the age are important predictors of the accuracy of screening by mammography. The sensitivity ranges from 63% in women with extremely dense breasts to 87% in women with almost entirely fatty breasts. The sensitivity increases with age from 69% in women of about 40 years of age to 83% in women 80 years and older (Carney, P. A., et al., Ann. Intern. Med. 138 (3) (2003) 168-175). Only 20-25% of mammographically detected abnormalities that are biopsied prove to be malignant. The visualization of precancerous and cancerous lesions represents the best approach to early detection, but mammography is an expensive test that requires great care and expertise both to perform and in the interpretation of results (WHO, Screening for Breast Cancer, May 10, 2002; Esserman, L., et al., J. Natl. Cancer Inst. 94 (2002) 369-375).

In the recent years a tremendous amount of so-called breast specific or even so-called breast cancer specific genes has been reported. The vast majority of the corresponding research papers or patent applications are based on data obtained by analysis of RNA expression patterns in breast (cancer) tissue versus a different tissue or an adjacent normal tissue, respectively. Such approaches may be summarized as differential mRNA display techniques.

As an example for data available from mRNA-display techniques, WO 00/60076 shall be mentioned and discussed. This application describes and claims more than two hundred isolated polynucleotides and the corresponding polypeptides as such, as well as their use in the detection of BC. However, it is general knowledge that differences on the level of mRNA are not mirrored by the level of the corresponding proteins. A protein encoded by a rare mRNA may be found in very high amounts and a protein encoded by an abundant mRNA may nonetheless be hard to detect and find at all (Chen, G., et al., Mol. Cell. Proteomics 1 (2002) 304-313). This lack of correlation between mRNA-level and protein level is due to reasons like mRNA stability, efficiency of translation, stability of the protein, etc.

There also are recent approaches investigating the differences in protein patterns between different tissues or between healthy and diseased tissue in order to identify candidate marker molecules which might be used in the diagnosis of BC. Wulfkuhle, J. D., et al., Cancer Res. 62 (2002) 6740-6749 have identified fifty-seven proteins which were differentially expressed between BC tissue and adjacent normal tissue. No data from liquid samples obtained from an individual are reported.

WO 02/23200 reports about twelve breast cancer-associated spots as found by surface-enhanced laser desorption and ionization (SELDI). These spots are seen more frequently in sera obtained from patients with BC as compared to sera obtained from healthy controls. However, the identity of the molecule(s) comprised in such spot, e.g their sequence, is not known.

Nipple aspirate fluid (NAF) has been used for many years as a potential non-invasive method to identify breast cancer-specific markers. Kuerer et al. compared bilateral matched pair nipple aspirate fluids from women with unilateral invasive breast carcinoma by 2D gel electrophoresis (Kuerer, H. M., et al., Cancer 95 (2002) 2276-2282). 30 to 202 different protein spots were detected in the NAF of breasts suffering from breast carcinoma and not in the matched NAF of the healthy breasts. These spots were detected by a gel image analysis. But the identity of the protein spots is not known.

Despite the large and ever growing list of candidate protein markers in the field of BC, to date clinical/diagnostic utility of these molecules is not known. In order to be of clinical utility a new diagnostic marker as a single marker should be at least as good as the best single marker known in the art. Or, a new marker should lead to a progress in diagnostic sensitivity and/or specificity either if used alone or in combination with one or more other markers, respectively. The diagnostic sensitivity and/or specificity of a test is best assessed by its receiver-operating characteristics, which will be described in detail below.

At present, only diagnostic blood tests based on the detection of cancer antigen 15-3 (CA 15-3), a tumor-associated mucin, and carcinoembryonic antigen (CEA), a tumor associated glycoprotein, are available to assist diagnosis in the field of BC. CA 15-3 is usually increased in patients with advanced breast cancer. CA 15-3 levels are rarely elevated in women with early stage breast cancer (Duffy, M. J., Crit. Rev. Clin. Lab. Sci. 38 (2001) 225-262). Cancers of the ovary, lung and prostate may also raise CA 15-3 levels. Elevated levels of CA 15-3 may be associated with non-cancerous conditions, such as benign breast or ovary disease, endometriosis, pelvic inflammatory disease, and hepatitis. Pregnancy and lactation can also cause CA 15-3 levels to raise (National Cancer Institute, Cancer Facts, Fact Sheet 5.18 (1998) 1-5). The primary use of CEA is in monitoring colon cancer, especially when the disease has metastasized. However, a variety of cancers can produce elevated levels of CEA, including breast cancer.

Due to the lack of organ and tumor specificity, neither measurement of CA 15-3 nor measurement of CEA are recommended for screening of BC. These tumor markers are helpful diagnostic tools in follow-up care of BC patients (Untch, M., et al., J. Lab. Med. 25 (2001) 343-352).

Whole blood, serum, plasma, or nipple aspirate fluid are the most widely used sources of sample in clinical routine. The identification of an early BC tumor marker that would allow reliable cancer detection or provide early prognostic information could lead to a diagnostic assay that would greatly aid in the diagnosis and in the management of this disease. Therefore, an urgent clinical need exists to improve the in vitro assessment of BC. It is especially important to improve the early diagnosis of BC, since for patients diagnosed early on chances of survival are much higher as compared to those diagnosed at a progressed stage of disease.

It was the task of the present invention to investigate whether a new marker can be identified which may be used in assessing BC.

Surprisingly, it has been found that use of the marker PDX1 can at least partially overcome the problems known from the state of the art.

### SUMMARY OF THE INVENTION

The present invention therefore relates to a method for assessing breast cancer comprising the steps of a) providing a liquid sample obtained from an individual, b) contacting said sample with a specific binding agent for PDX1 under conditions appropriate for formation of a complex between said binding agent and PDX1, and c) correlating the amount of complex formed in (b) to the assessment of breast cancer

Another preferred embodiment of the invention is a method for assessing breast cancer comprising the steps of a) contacting a liquid sample obtained from an individual with a specific binding agent for PDX1 under conditions appropriate for formation of a complex between said binding agent and PDX1, and b) correlating the amount of complex formed in (a) to the assessment of breast cancer.

Yet another preferred embodiment of the invention relates to a method for assessing breast cancer in vitro by biochemical markers, comprising measuring in a sample the concentration of PDX1 and of one or more other marker of breast cancer and using the concentrations determined in the assessment of breast cancer.

The present invention also relates to the use of a marker panel comprising at least PDX1 and CA 15-3 in the assessment of BC.

The present invention also relates to the use of a marker panel comprising at least PDX1 and CEA in the assessment of BC.

The present invention also relates to the use of a marker panel comprising at least PDX1 and CRABP-II in the assessment of BC.

The present invention also relates to the use of a marker panel comprising at least PDX1 and ASC in the assessment of BC.

The present invention also provides a kit for performing the method according to the present invention comprising at least the reagents required to measure PDX1 and CA 15-3, respectively, and optionally auxiliary reagents for performing the measurement.

The present invention also provides a kit for performing the method according to the present invention comprising at least the reagents required to measure PDX1 and CEA, respectively, and optionally auxiliary reagents for performing the measurement.

In a further preferred embodiment the present invention relates to a method for assessing breast cancer in vitro comprising measuring in a sample the concentration of a) PDX1, b) optionally one or more other marker of breast cancer, and c) using the concentrations determined in step (a) and optionally step (b) in the assessment of breast cancer.

### DETAILED DESCRIPTION OF THE INVENTION

As used herein, each of the following terms has the meaning associated with it in this section.

The term “marker” or “biochemical marker” as used herein refers to a molecules to be used as a target for analyzing patient test samples. Examples of such molecular targets are proteins or polypeptides themselves as well as antibodies present in a sample. Proteins or polypeptides used as a marker in the present invention are contemplated to include any variants of said protein as well as fragments of said protein or said variant, in particular, immunologically detectable fragments. One of skill in the art would recognize that proteins which are released by cells or present in the extracellular matrix which become damaged, e.g., during inflammation could become degraded or cleaved into such fragments. Certain markers are synthesized in an inactive form, which may be subsequently activated by proteolysis. As the skilled artisan will appreciate, proteins or fragments thereof may also be present as part of a complex. Such complex also may be used as a marker in the sense of the present invention. Variants of a marker polypeptide are encoded by the same gene, but differ in their PI or MW, or both (e.g., as a result of alternative mRNA or pre-mRNA processing, e.g. alternative splicing or limited proteolysis) and in addition, or in the alternative, may arise from differential post-translational modification (e.g., glycosylation, acylation, and/or phosphorylation).

The term “assessing breast cancer” is used to indicate that the method according to the present invention will (alone or together with other markers or variables, e.g., the criteria set forth by the UICC (UICC (International Union Against Cancer), Sobin, L. H., Wittekind, Ch. (eds), TNM Classification of Malignant Tumours, fifth edition, 1997)) e.g., aid the physician to establish or confirm the absence or presence of BC or aid the physician in the prognosis, the detection of recurrence (follow-up of patients after surgery) and/or the monitoring of treatment, especially of chemotherapy.

The term “sample” as used herein refers to a biological sample obtained for the purpose of evaluation in vitro. In the methods of the present invention, the sample or patient sample preferably may comprise any body fluid. Preferred test samples include blood, serum, plasma, nipple aspirate fluid, urine, saliva, and synovial fluid. Preferred samples are whole blood, serum, plasma or nipple aspirate fluid, with plasma or serum being most preferred. As the skilled artisan will appreciate, any such assessment is made in vitro. The patient sample is discarded afterwards. The patient sample is solely used for the in vitro method of the invention and the material of the patient sample is not transferred back into the patient's body. Typically, the sample is a liquid sample, e.g., whole blood, serum, or plasma.

In a preferred embodiment the present invention relates to a method for assessing BC in vitro by biochemical markers, comprising measuring in a sample the concentration of PDX1 and using the concentration determined in the assessment of BC.

The protein PDX1 (also known as peroxiredoxin 1, thioredoxin peroxidase 2, thioredoxin-dependent peroxide reductase 2, proliferation-associated protein pag, natural killer cell enhancing factor A (nkef-A); natural killer-enhancing factor A; Swiss-PROT: Q06830) is characterized by the sequence given SEQ ID No. 1 or its isoforms. This sequence translates to a molecular weight of 22,110 Da.

PDX1 is known to the art from the following publications: Chang, J. W., et al., Biochem. Biophys. Res. Commun. 289 (2) (2001) 507-512; Noh, D. Y., et al., Anticancer Res. 21 (3B) (2001) 2085-2090; Yanagawa, T., et al., Cancer Lett. 156 (1) (2000) 27-35. PDX1 may play an antioxidant protective role in cells and may contribute to the antiviral activity of CD8(+) T-cells. This protein may have a proliferative effect and play a role in cancer development or progression. The peroxiredoxins (Prx) are a family of 25 kDa peroxidases that can reduce H₂O₂ using an electron from thioredoxin (Trx) or other substances.

As obvious to the skilled artisan, the present invention shall not be construed to be limited to the full-length protein PDX1 of SEQ ID NO:1. Physiological or artificial fragments of PDX1, secondary modifications of PDX1, as well as allelic variants of PDX1 are also encompassed by the present invention. In this regard an “allelic variant” is understood to represent the gene product of one of two or more different forms of a gene or DNA sequence that can exist at a genetic single locus. Artificial fragments preferably encompass a peptide produced synthetically or by recombinant techniques, which at least comprises one epitope of diagnostic interest consisting of at least 6 contiguous amino acids as derived from the sequence disclosed in SEQ ID NO:1. Such a fragment may advantageously be used for generation of antibodies or as a standard in an immunoassay. More preferred the artificial fragment comprises at least two epitopes of interest appropriate for setting up a sandwich immunoassay. Preferably, full-length PDX1 or a physiological variant of this marker is detected in a method according to the present invention.

The assessment method according to the present invention is based on a liquid sample which is derived from an individual. Unlike to methods known from the art PDX1 is measured from this liquid sample by use of a specific binding agent. A specific binding agent is, e.g., a receptor for PDX1, a lectin binding to PDX1 or an antibody to PDX1. A specific binding agent has at least an affinity of 10⁷ l/mol for its corresponding target molecule. The specific binding agent preferably has an affinity of 10⁸ l/mol or even more preferred of 10⁹ l/mol for its target molecule. As the skilled artisan will appreciate the term specific is used to indicate that other biomolecules present in the sample do not significantly bind to the binding agent specific for PDX1. Preferably, the level of binding to a biomolecule other than the target molecule results in a binding affinity which is only 10%, more preferably only 5% of the affinity of the target molecule or less. A most preferred specific binding agent will fulfill both the above minimum criteria for affinity as well as for specificity.

A specific binding agent preferably is an antibody binding to PDX1. The term antibody refers to a polyclonal antibody, a monoclonal antibody, fragments of such antibodies, as well as to genetic constructs comprising the binding domain of an antibody.

Any antibody fragment retaining the above criteria of a specific binding agent can be used. Antibodies are generated by state of the art procedures, e.g., as described in Tijssen (Tijssen, P., Practice and theory of enzyme immunoassays 11 (1990) the whole book, especially pages 43-78; Elsevier, Amsterdam). In addition, the skilled artisan is well aware of methods based on immunosorbents that can be used for the specific isolation of antibodies. By these means the quality of polyclonal antibodies and hence their performance in immunoassays can be enhanced. (Tijssen, P., supra, pages 108-115).

For the achievements as disclosed in the present invention polyclonal antibodies raised in rabbits have been used. However, clearly also polyclonal antibodies from different species, e.g. rats or guinea pigs, as well as monoclonal antibodies can also be used. Since monoclonal antibodies can be produced in any amount required with constant properties, they represent ideal tools in development of an assay for clinical routine. The generation and use of monoclonal antibodies to PDX1 in a method according to the present invention is yet another preferred embodiment.

The diagnostic method according to the present invention is based on a liquid sample which is derived from an individual. Unlike to methods known from the art PDX1 is measured from this liquid sample by use of a specific binding agent.

As the skilled artisan will appreciate now, that PDX1 has been identified as a marker which is useful in the assessment of BC, alternative ways may be used to reach a result comparable to the achievements of the present invention. For example, alternative strategies to generate antibodies may be used. Such strategies comprise amongst others the use of synthetic peptides, representing an epitope of PDX1 for immunization. Preferably, a synthetic peptide comprises a subsequence of SEQ ID NO:1 which is specific for PDX1, i.e., which has a comparatively low homology to other/related polypeptides. It is preferred that the synthetic peptide comprises a contiguous subsequence consisting of 5 to 25 amino acid residues of SEQ ID NO:1. More preferred, the peptide comprises a contiguous subsequence consisting of 10 to 15 amino acid residues of SEQ ID NO:1.

Alternatively, DNA immunization also known as DNA vaccination may be used.

For measurement the liquid sample obtained from an individual is incubated with the specific binding agent for PDX1 under conditions appropriate for formation of a binding agent PDX1-complex. Such conditions need not be specified, since the skilled artisan without any inventive effort can easily identify such appropriate incubation conditions.

As a final step according to the method disclosed in the present invention the amount of complex is measured and correlated to the diagnosis of BC. As the skilled artisan will appreciate there are numerous methods to measure the amount of the specific binding agent PDX1-complex, all described in detail in relevant textbooks (cf., e.g., Tijssen P., supra, or Diamandis et al., eds. (1996) Immunoassay, Academic Press, Boston).

Preferably PDX1 is detected in a sandwich type assay format. In such assay a first specific binding agent is used to capture PDX1 on the one side and a second specific binding agent, which is labeled to be directly or indirectly detectable is used on the other side.

As mentioned above, it has surprisingly been found that PDX1 can be measured from a liquid sample obtained from an individual sample. No tissue and no biopsy sample is required to apply the marker PDX1 in the diagnosis of BC.

In a preferred embodiment the method according to the present invention is practiced with serum as liquid sample material. In a further preferred embodiment the method according to the present invention is practiced with plasma as liquid sample material. In a further preferred embodiment the method according to the present invention is practiced with whole blood as liquid sample material. In a further preferred embodiment the method according to the present invention is practiced with nipple aspirate fluid as liquid sample material.

The inventors of the present invention have surprisingly been able to detect protein PDX1 in a bodily fluid sample. Even more surprising they have been able to demonstrate that the presence of PDX1 in such liquid sample obtained from an individual can be correlated to the diagnosis of breast cancer. Preferably, an antibody to PDX1 is used in a qualitative (PDX1 present or absent) or quantitative (PDX1 amount is determined) immunoassay.

In the assessment of BC especially the following intended uses are considered important.

Screening:

BC is one of the most frequent cancers among women in developed countries. Because of its high prevalence, its long asymptomatic phase and the presence of premalignant lesions, BC meets many of the criteria for screening. Clearly, a serum tumor marker which has acceptable sensitivity and specificity would be more suitable for screening than established methods. In a preferred embodiment the diagnostic method according to the present invention is used for screening purposes. I.e., it is used to assess subjects without a prior diagnosis of BC by measuring the level of PDX1 and correlating the level measured to the presence or absence of BC.

It is conceivable that PDX1 alone will not suffice to allow for a general screening e.g. of the risk population for BC. Most likely no single biochemical marker in the circulation will ever meet the sensitivity and specificity criteria required for screening purposes. Rather it has to be expected that a marker panel will have to be used in BC screening. Thus, the marker PDX1 will form an integral part of a marker panel appropriate for screening purposes. The present invention therefore relates to the use of PDX1 as one marker of a BC marker panel for BC screening purposes.

Diagnostic Aid

The inventors also contemplate PDX1 to be used as a diagnostic aid, especially by establishing a baseline value to indicate tumor load before breast surgery. The present invention thus also relates to the use of PDX1 for establishing a baseline value before surgery for BC. Antibodies to PDX1 with great advantage can also be used in established procedures, e.g., to detect breast cancer cells in situ, in biopsies, or in immunohistological procedures.

Prognosis

As PDX1 alone contributes to the differentiation of BC patients from healthy controls or from healthy controls plus non-malignant diseases, it has to be expected that it will aid in assessing the prognosis of patients suffering from BC. The level of preoperative PDX1 will most likely be combined with one or more other marker for BC and/or the TNM staging system. In a preferred embodiment PDX1 is used in the prognosis of patients with BC.

Monitoring of Therapy

The inventors furthermore contemplate that PDX1 will be a clinically useful marker for monitoring of chemotherapy, radiotherapy or immune therapy. Increased levels of PDX1 are directly correlated to tumor burden. For example, after chemotherapy a short term (few hours to 14 days) increase in PDX1 may serve as an indicator of tumor cell death. The present invention therefore also relates to the use of PDX1 in the monitoring of BC patients under chemotherapy. In addition, the present invention therefore also relates to the use of PDX1 in the monitoring of BC patients under radiotherapy. Furthermore, the present invention relates to the use of PDX1 in the monitoring of BC patients under immune therapy.

Follow-Up

A number of patients who undergo surgical resection aimed at cure, later develop recurrent of metastatic disease. Since recurrent/metastatic disease is invariably fatal, considerable research has focused on its identification at an early and thus potentially treatable stage. Consequently, many of these patients undergo a postoperative surveillance program.

The follow-up of patients with BC after surgery is one of the most important fields of use for an appropriate biochemical marker. In the follow-up (from 3 months to 10 years) an increase of PDX1 can be used as an indicator for tumor recurrence. Due to the high sensitivity of PDX1 in the BC patients investigated it is expected that PDX1 alone or in combination with one or more other marker will be of great help in the follow-up of BC patients, especially in BC patients after surgery. The use of a marker panel comprising PDX1 and one or more other marker of BC in the follow-up of BC patients represents a further preferred embodiment of the present invention.

Measuring the level of protein PDX1 has proven very advantageous in the field of BC. Therefore, in a further preferred embodiment, the present invention relates to use of protein PDX1 as a marker molecule in the diagnosis of breast cancer from a liquid sample obtained from an individual.

The ideal scenario for diagnosis would be a situation wherein a single event or process would cause the respective disease as, e.g., in infectious diseases. In all other cases correct diagnosis can be very difficult, especially when the etiology of the disease is not fully understood as is the case of BC. As the skilled artisan will appreciate, no biochemical marker, for example in the field of BC, is diagnostic with 100% specificity and at the same time 100% sensitivity for a given disease. Rather, biochemical markers are used to assess with a certain likelihood or predictive value the presence or absence of a disease. Therefore, in routine clinical diagnosis various clinical symptoms and biological markers are generally considered together in the diagnosis, treatment, and management of the underlying disease.

Biochemical markers can either be determined individually or, in a preferred embodiment of the invention, they can be measured simultaneously using a chip- or a bead-based array technology. The concentrations of the biomarkers are then interpreted independently using an individual cut-off for each marker or they are combined for interpretation.

The use of protein PDX1 itself, represents a significant progress to the challenging field of BC diagnosis. Combining measurements of PDX1 with other known markers, e.g. CA 15-3 and CEA, or with other markers of BC presently known or yet to be discovered, leads to further improvements.

Recently, novel markers with clinical utility to assess BC have been discovered. They include cellular retinoic acid-binding protein II (CRABP-II) and the apoptosis-associated speck-like protein containing a caspase-associated recruitment domain (ASC).

Cellular Retinoic Acid-Binding Protein II

The cellular retinoic acid-binding protein II (CRABP-II) (Swiss-PROT: P29373) is one of two isoforms presently known. The two isoforms (CRABP-I and -II) were first characterized by Siegenthaler et al. 1992. CRABP-II was shown to be the major isoform, highly expressed in human epidermis by fibroblasts and keratinocytes (Siegenthaler, G., Biochemical Journal 287 (1992) 383-389).

An increased concentration of CRABP-II was found in keratoacanthoma and squamous cell cancer but not in basal cell carcinoma of the skin by Vahlquist et al. (Vahlquist, A., et al., J. Invest. Dermatol. 106 (1996) 1070-1074).

In the cytoplasm, CRABP-II regulates the intracellular retinoic acid (RA) concentration, transport, and metabolism. It has been demonstrated that RA induced CRABP-II mRNA levels 2 fold in squamous cell cancer by transcriptional upregulation (Vo, H. P., Crowe, D. L., Anticancer Res. 18 (1998) 217-224).

The presence of CRABP-II in human breast cancer cells was first described by Wang et al. 1998. They localized CRABP-II in human breast cancer cells by immunohistochemistry (Wang, Y., et al., Laboratory Investigation 78 (1998) 30 A).

The function of CRABP-II in mammary carcinoma cells was described by Budhu, A. S., and Noy, N. (Mol. Cell. Biol. 22 (2002) 2632-2641). The cytosolic CRABP-II undergoes a nuclear localization upon binding RA and interacts with retinoic acid receptor (RAR) by building a short lived CRABP-II-RAR-complex. The overexpression of CRABP-II in MCF7 mammary cell lines enhances their sensitivity to retinoic acid-induced growth inhibition (Budhu, A. S., Noy, N., supra).

In a first proteomics analysis of matched normal ductal/lobular units and ductal carcinoma in situ (DCIS) of the human breast Wulfkuhle et al. (Cancer Res. 62 (2002) 6740-6749) identified fifty-seven proteins that were differentially expressed in normal and precancerous cells. The level of CRABP-II was reported to be five times higher in DCIS than in normal cells. A comparable increase has been reported for as many as 23 proteins. But no further investigations were carried out, e.g. whether CRABP-II could be detected in liquid samples (Wulfkuhle, J. D. et al., supra).

The clinical utility of CRABP-II for assessing BC has recently been described in WO 2004/111650.

Apoptosis-Associated Speck-Like Protein Containing a Caspase-Associated Recruitment Domain

The “apoptosis-associated speck-like protein containing a caspase-associated recruitment domain” (ASC) is also known as “target of methylation-induced silencing 1” (TMS1) (Swiss-PROT: Q9ULZ3).

Caspase-associated recruitment domains (CARDs) mediate the interaction between adaptor proteins such as APAFL (apoptotic protease activating factor 1) and the pro-form of caspases (e.g., CASP 9) participating in apoptosis. ASC is a member of the CARD-containing adaptor protein family.

By immunoscreening a promyelocytic cell line, Masumoto et al. isolated a cDNA encoding ASC. The deduced 195-amino acid protein contains an N-terminal pyrin-like domain (PYD) and an 87-residue C-terminal CARD. Western blot analysis showed expression of a 22-kDa protein and indicated that ASC may have proapoptotic activity by increasing the susceptibility of leukemia cell lines to apoptotic stimuli by anticancer drugs (Masumoto, J., et al., J. Biol. Chem. 274 (1999) 33835-33838).

Methylation-sensitive restriction PCR and methylation-specific PCR (MSP) analyses by Conway et al. indicated that silencing of ASC correlates with hypermethylation of the CpG island surrounding exon1 and that overexpression of DNMT1 (DNA cytosine-5-methyltransferase-1) promotes hypermethylation and silencing of ASC. Breast cancer cell lines, but not normal breast tissue, exhibited complete methylation of ASC and expressed no ASC message. Expression of ASC in breast cancer cell lines inhibited growth and reduced the number of surviving colonies. Conway et al. concluded that ASC functions in the promotion of caspase-dependent apoptosis and that overexpression of ASC inhibits the growth of breast cancer cells (Conway, K. E., et al., Cancer Res. 60 (2000) 6236-6242).

McConnell and Vertino showed that inducible expression of ASC inhibits cellular prolifertion and induces DNA fragmentation that can be blocked by caspase inhibitor. Immunofluorescence microscopy demonstrated that induction of apoptosis causes a CARD-dependent shift from diffuse cytoplasmic expression to spherical perinuclear aggregates (McConnell, B. B., and Vertino, P. M., Cancer Res. 60 (2000) 6243-6247).

Moriani et al. observed methylation of ASC gene not only in breast cancer cells but also in gastric cancer. They suggested a direct role for aberrant methylation of the ASC gene in the progression of breast and gastric cancer involving down-regulation of the proapoptotic ASC gene (Moriani, R., et al., Anticancer Res. 22 (2002) 4163-4168).

Conway et al. examined primary breast tissues for TMS1 methylation and compared the results to methylation in healthy tissues (Conway, K. E., et al., Cancer Res. 60 (2000) 6236-6242). Levine et al. found that ASC silencing was not correlated with methylation of specific CpG sites, but rather was associated with dense methylation of ASC CpG island. Breast tumor cell lines containing exclusively methylated ASC copies do not express ASC, while in partially methylated cell lines the levels of ASC expression are directly related to the percentage of methylated ASC allels present in the cell population (Levine, J. J., et al., Oncogene 22 (2003) 3475-3488).

Virmani et al. examined the methylation status of ASC in lung cancer and breast cancer tissue. They found that aberrant methylation of ASC was present in 46% of breast cancer cell lines and in 32% of breast tumor tissue. Methylation was rare in non-malignant breast tissue (7%) (Virmani, A., et al., Int. J. Cancer 106 (2003) 198-204). Shiohara et al. found out that up-regulation of ASC is closely associated with inflammation and apoptosis in human neutrophils (Shiohara, M., et al., Blood 98 (2001) 229a). Masumoto et al. observed high levels of ASC abundantly expressed in epithelial cells and leucocytes (Masumoto, J., et al., J. Histochem. Cytochem. 49 (2001) 1269-1275).

The clinical utility of ASC for assessing BC has recently been described in WO 2005/040806.

Therefore in a further preferred embodiment the present invention relates to the use of PDX1 as a marker molecule for breast cancer in combination with one or more marker molecules for breast cancer in the diagnosis of breast cancer from a liquid sample obtained from an individual. In this regard, the expression “one or more” denotes 1 to 10, preferably 1 to 5, more preferred 3 or 4. Preferred selected other BC markers with which the measurement of PDX1 may be combined are CEA, CA 15-3, CRABP-II, and ASC. Most preferred, PDX1 is used as part of a marker panel at least comprising PDX1 and a marker selected from the group consisting of CEA, CA 15-3, CRABP-II, and ASC.

In a further preferred embodiment of the invention the assessment of breast cancer according to the present invention is performed in a method comprising measuring in a sample the concentration of a) PDX1, b) optionally one or more other marker of breast cancer, and c) using the concentration determined in step (a) and optionally step (b) in the assessment of breast cancer.

The present invention is also directed to a method for assessing BC in vitro by biochemical markers, comprising measuring in a sample the concentration of PDX1 and of one or more other marker of BC and using the concentrations determined in the assessment of BC.

Preferably the method for assessment of BC is performed by measuring the concentration of PDX1 and of one or more other marker and by using the concentration of PDX1 and of the one or more other marker in the assessment of BC.

Preferably, the method according to the present invention is used with samples of patients suspected to be suffering from breast cancer. An individual suspected of suffering from breast cancer is an individual for which other types of cancers have been excluded. Other cancers include but are not limited to cancers of the colon, lung, stomach, ovary, and prostate. A preferred embodiment of the invention is therefore a method for the diagnosis of breast cancer comprising the steps of a) providing a liquid sample obtained from an individual suspected of suffering from breast cancer, b) contacting said sample with a specific binding agent for PDX1 under conditions appropriate for formation of a complex between said binding agent and PDX1, and c) correlating the amount of complex formed in (b) to the diagnosis of breast cancer.

Diagnostic reagents in the field of specific binding assays, like immunoassays, usually are best provided in the form of a kit, which comprises the specific binding agent and the auxiliary reagents required to perform the assay. The present invention therefore also relates to an immunological kit comprising at least one specific binding agent for PDX1 and auxiliary reagents for measurement of PDX1.

Accuracy of a test is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot. By convention, this area is always ≧0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

Clinical utility of the novel marker PDX1 is best assessed in comparison to and in combination with the established marker CA 15-3 using a receiver operator curve analysis (ROC; Zweig, M. H., and Campbell, G., Clin. Chem. 39 (1993) 561-577). This analysis is based on well-defined patient cohorts consisting of 50 samples each from patients with invasive ductal or lobular carcinoma in T1-3; N0; M0, more progressed tumor, i.e., T4 and/or various severity of metastasis (N+ and/or M+), medullary, papillary, mucinous and tubular carcinoma, ductal carcinoma in situ, and healthy controls, respectively.

Combining measurements of PDX1 with other recently discovered markers or with known markers like CEA and CA 15-3, or with other markers of BC yet to be discovered, leads and will lead, respectively, to further improvements in assessment of BC.

The following examples, references, sequence listing and figure are provided to aid the understanding of the present invention, the true scope of which is set forth in the appended claims. It is understood that modifications can be made in the procedures set forth without departing from the spirit of the invention.

### ABBREVIATIONS

ABTS 2,2′-azino-di-[3-ethylbenzthiazoline sulfonate (6)] diammonium salt
BSA bovine serum albumin
cDNA complementary DNA
CHAPS (3-[(3-cholamidopropyl)-dimethylammonio]-1-propane-sulfonate)
DMSO dimethyl sulfoxide
DTT dithiothreitol
EDTA ethylene diamine tetraacetic acid
ELISA enzyme-linked immunosorbent assay
HRP horseradish peroxidase
IAA iodacetamid
IgG immunoglobulin G
IEF isoelectric focusing
IPG immobilized pH gradient
LDS lithium dodecyl sulfate
MALDI-TOF matrix-assisted laser desorption/ionization-time of flight mass spectrometry
MES mesity, 2,4,6-trimethylphenyl
OD optical density
PAGE polyacrylamide gel electrophoresis
PBS phosphate buffered saline
PI isoelectric point
RTS rapid translation system
SDS sodium dodecyl sulfate

### Specific embodiments

### EXAMPLE 1

### Identification of PDX1 as a Potential Breast Cancer Marker

Sources of Tissue

In order to identify tumor-specific proteins as potential diagnostic markers for breast cancer, analysis of two different kinds of tissue is performed using proteomics methods.

In total, tissue specimen from 10 patients suffering from breast cancer are analyzed. From each patient two different tissue types are collected from therapeutic resections: tumor tissue (>80% tumor) (T), and adjacent healthy tissue (N). The latter tissue type serves as matched healthy control sample. Tissues are immediately snap frozen after resection and stored at −80° C. before processing. Tumors are diagnosed by histopathological criteria.

Tissue Preparation

0.8-1.2 g of frozen tissue are put into a mortar and completely frozen by liquid nitrogen. The tissue is pulverized in the mortar, dissolved in the 10-fold volume (w/v) of lysis buffer (40 mM Na-citrate, 5 mM MgCl₂, 1% Genapol X-080, 0.02% Na-azide, Complete® EDTA-free [Roche Diagnostics GmbH, Mannheim, Germany, Cat. No. 1 873 580]) and subsequently homogenized in a Wheaton® glass homogenizer (20×loose fitting, 20×tight fitting). 3 ml of the homogenate are subjected to a sucrose-density centrifugation (10-60% sucrose) for 1 h at 4,500×g. After this centrifugation step three fractions are obtained. The fraction on top of the gradient contains the soluble proteins and is used for further analysis.

Immobilization of Monoclonal Antibody Anti-Human Albumin on CNBr-Activated Sepharose 4B

Freeze-dried CNBr-activated Sepharose 4B (Amersham Biosciences, 17-0430-01) is reswollen and washed according to the instructions of the manufacturer. Monoclonal antibody directed against human albumin is dissolved in 0.1 M NaHCO₃, pH 8.3, 0.5 M NaCl, 10 mg/ml. 1 ml antibody solution is mixed with 1 ml reswollen CNBr-activated Sepharose 4B. The reaction time is 1 h. Blocking of the remaining active groups and washing of the gel is carried out according to the instructions of the manufacturer.

Depletion of Serum Albumin

7 ml anti-albumin gel is equilibrated in lysis buffer without Genapol X-080. 7 ml of the upper fraction of the sucrose-density centrifugation (see above, tissue preparation) are applied onto the column and washed through with lysis buffer without Genalpol X-080. The combined effluent is used for further analysis.

Sample Preparation for LC-ESI-MSMS-Analysis

The protein concentration of the soluble protein fraction is determined using Bio-Rad® protein assay (Cat. No. 500-0006; Bio-Rad Laboratories GmbH, München, Germany) following the instructions of the supplier's manual. To a volume corresponding to 200 μg of protein 4 ml reduction buffer (9 M urea, 2 mM DTT, 100 mM KH₂PO₄, pH 8.2 NaOH) is added and incubated for 1 h. The solution is concentrated to 250 μl in an Amicon® Ultra 10 kD device (Millipore GmbH, Schwalbach, Germany). For alkylation the 250 μl are transferred into 1 ml alkylation buffer (9 M urea, 4 mM iodoacetamide, 100 mM KH₂PO₄, pH 8.2 NaOH), incubated for 6 h and subsequently concentrated in an Amicon® Ultra 10 kD device to 250 μl. For washing 1 ml 9 M urea is added and again concentrated in an Amicon® Ultra 10 kD device to 250 μl. Washing is repeated three-times.

For protease digestion the concentrated solution is diluted to 2.5 M urea and incubated with 4 μg trypsin (Proteomics grade, Roche Diagnostics GmbH, Mannheim, Germany) over night. The digestion is stopped by adding 1 ml 1% formic acid and analyzed.

LC-ESI-MSMS-Analysis

The tryptic digest (500 μl) is separated on a two-dimensional Nano-HPLC-System (Ultimate, Famos, Switchos; LC Packings, Idstein, Germany) consisting of a SCX and a RP Pepmep C18 column (LC Packings, Idstein, Germany). The 11 SCX fractions (step elution with 0, 5, 10, 25, 50, 100, 200, 300, 400, 500, 1,500 mM NH₄Ac) are successively further separated on the RP column with a 90 min gradient (5-95% acetonitrile) and analyzed online using data dependent scans with an ESI-MS ion trap (LCQ deca XP; Thermo Electron, Massachusetts, USA; see Table 2 for parameters). For each sample three runs are performed. The raw data are processed with Bioworks 3.1 software (Thermo Electron, Massachusetts, USA) using the parameters listed in Table 2. The resulting lists of identified peptides and proteins from replicate runs where combined.

The protein PDX1 is identified with the sequences given in Table 1.

Detection of PDX1 as a Potential Marker for Breast Cancer

For each patient the identified proteins and the number of corresponding peptides from the tumor sample are compared to the accordant results from adjacent normal tissue. By this means, protein PDX1 is found specifically expressed or strongly overexpressed in tumor tissue. It therefore—amongst many other proteins—qualifies as a candidate marker for use in the diagnosis of breast cancer.

TABLE 1
Sequences of protein PDX1 which were identified with
Bioworks 3.1 from LCQ-MS2-data:

| | |
|---|---|
| i | GLFIIDDK (SEQ ID NO: 2) |
| ii | GLFIIDDKGILR (SEQ ID NO: 3) |
| iii | KLNCQVIGASVDSHFCHLAWVNTPK (SEQ ID NO: 4) |
| iv | KQGGLGPMNIPLVSDPK (SEQ ID NO: 5) |
| v | LNCQVIGASVDSHFCHLAWVNTPK (SEQ ID NO: 6) |
| vi | LVQAFQFTDK (SEQ ID NO: 7) |
| vii | TIAQDYGVLKADEGISFR (SEQ ID NO: 8) |

TABLE 2
MSMS-data acquisition and Bioworks 3.1 search parameters

| | | |
|---|---|---|
| MSMS-data | MS exclusion | 350-2,000 Da for precursor ions |
| acquisition | | |
| | Repeat count | 2 |
| | Repeat duration | 0.25 min |
| | Exclusion list size | 25 |
| | Exclusion duration | 5 min |
| | Exclusion mass width | low 0.5 Da, high 1.5 Da |
| Bioworks | Number of ions | 35 |
| | Minimal ion intensity | 100,000 counts |
| | Precursor mass | 1.2 Da |
| | tolerance | |
| | Fragment mass | 1.4 Da |
| | tolerance | |
| | X_{corr} | >2; 2.5; 3 (z = 1; 2; 3) |
| | dCn | >0.1 |
| | Sp | >500 |
| Databases | | Swissprot; Humangp |
| | | (assembled by Roche |
| | | Bioinformatics) |

### EXAMPLE 2

### Generation of Antibodies to the Breast Cancer Marker Protein PDX1

Polyclonal antibody to the breast cancer marker protein PDX1 is generated for further use of the antibody in the measurement of serum and plasma and blood levels of PDX1 by immunodetection assays, e.g. Western Blotting and ELISA

Recombinant Protein Expression and Purification

In order to generate antibodies to PDX1, recombinant expression of the protein is performed for obtaining immunogens. The expression is done applying a combination of the RTS 100 expression system and E. coli. In a first step, the DNA sequence is analyzed and recommendations for high yield cDNA silent mutational variants and respective PCR-primer sequences are obtained using the “ProteoExpert RTS E. coli HY” system. This is a commercial web-based service (www.proteoexpert.com). Using the recommended primer pairs, the “RTS 100 E. coli Linear Template Generation Set, His-tag” (Roche Diagnostics GmbH, Mannheim, Germany, Cat. No. 3186237) system to generate linear PCR templates from the cDNA for in-vitro transcription and expression of the nucleotide sequence coding for the PDX1 protein is used. For Western-blot detection and later purification, the expressed protein contains a His-tag. The best expressing variant is identified. All steps from PCR to expression and detection are carried out according to the instructions of the manufacturer. The respective PCR product, containing all necessary T7 regulatory regions (promoter, ribosomal binding site and T7 terminator) is cloned into the pBAD TOPO vector (Invitrogen, Karlsruhe, Germany, Cat. No. K 4300/01) following the manufacturer's instructions. For expression using the T7 regulatory sequences, the construct is transformed into E. coli BL 21 (DE 3) (Studier, F. W., et al., Methods Enzymol. 185 (1990) 60-89) and the transformed bacteria are cultivated in a 1 l batch for protein expression.

Purification of His-PDX1 fusion protein is done following standard procedures on a Ni-chelate column. Briefly, 1 l of bacteria culture containing the expression vector for the His-PDX1 fusion protein is pelleted by centrifugation. The cell pellet is resuspended in lysis buffer, containing phosphate, pH 8.0, 7 M guanidium chloride, imidazole and thioglycerole, followed by homogenization using a Ultra-Turrax. Insoluble material is pelleted by high speed centrifugation and the supernatant is applied to a Ni-chelate chromatographic column. The column is washed with several bed volumes of lysis buffer followed by washes with buffer, containing phosphate, pH 8.0 and urea. Finally, bound antigen is eluted using a phosphate buffer containing SDS under acid conditions.

Synthesis of Hemocyanin-Peptide-Conjugates for the Generation of Antibodies

Synthesis is carried out using heterobifunctional chemistry (maleimide/SH-chemistry). Selected cysteine containing PDX1-peptides are coupled to 3-maleimidohexanoyl-N-hydroxysuccinimidester (MHS) activated hemocyanin from Concholepas concholepas (Sigma, B-8556).

Hemocyanin is brought to 10 mg/ml in 100 mM NaH₂PO₄/NaOH, pH 7.2. Per ml hemocyanin 100 μl MHS (12.3 mg in DMSO) are added and incubated for 1 h. The sample is dialyzed over night against 100 mM NaH₂PO₄/NaOH, pH 6.5 and adjusted to 6 mg/ml with dialysis buffer. A selected cysteine containing PDX1-peptide is dissolved in DMSO (5 mg/ml for a peptide of 1500 Dalton). Per ml MHS-activated hemocyanin (6 mg/ml) 20 μl of 100 mM EDTA, pH 7.0 and 100 μl of the selected cysteine containing PDX1-peptide are added. After 1 h the remaining maleimide groups are blocked by the addition of 10 μl 0.5 M cysteine/HCl per ml reaction mixture. This preparation is used for immunization without further purification.

Production of Monoclonal Antibodies Against PDX1

a) Immunization of Mice

12 week old A/J mice are initially immunized intraperitoneally with 100 μg PDX1 or hemocyanin-peptide-conjugate (see above). This is followed after 6 weeks by two further intraperitoneal immunizations at monthly intervals. In this process each mouse is administered 100 μg PDX1 or hemocyanin-peptide-conjugate adsorbed to aluminium hydroxide and 10⁹ germs of Bordetella pertussis. Subsequently the last two immunizations are carried out intravenously on the 3rd and 2nd day before fusion using 100 μg PDX1 or hemocyanin-peptide-conjugate in PBS buffer for each.

b) Fusion and Cloning

Spleen cells of the mice immunized according to a) are fused with myeloma cells according to Galfre, G., and Milstein, C., Methods in Enzymology 73 (1981) 3-46. In this process ca. 1×10⁸ spleen cells of the immunized mouse are mixed with 2×10⁷ myeloma cells (P3×63-Ag8-653, ATCC CRL1580) and centrifuged (10 min at 300×g and 4° C.). The cells are then washed once with RPMI 1640 medium without fetal calf serum (FCS) and centrifuged again at 400×g in a 50 ml conical tube. The supernatant is discarded, the cell sediment is gently loosened by tapping, 1 ml PEG (molecular weight 4,000, Merck, Darmstadt) is added and mixed by pipetting. After 1 min in a water-bath at 37° C., 5 ml RPMI 1640 without FCS is added drop-wise at room temperature within a period of 4-5 min. Afterwards 5 ml RPMI 1640 containing 10% FCS is added drop-wise within ca. 1 min, mixed thoroughly, filled to 50 ml with medium (RPMI 1640+10% FCS) and subsequently centrifuged for 10 min at 400×g and 4° C. The sedimented cells are taken up in RPMI 1640 medium containing 10% FCS and sown in hypoxanthine-azaserine selection medium (100 mmol/l hypoxanthine, 1 μg/ml azaserine in RPMI 1640+10% FCS). Interleukin 6 at 100 U/ml is added to the medium as a growth factor.

After ca. 10 days the primary cultures are tested for specific antibody. PDX1-positive primary cultures are cloned in 96-well cell culture plates by means of a fluorescence activated cell sorter. In this process again interleukin 6 at 100 U/ml is added to the medium as a growth additive.

c) Immunoglobulin Isolation from the Cell Culture Supernatants

The hybridoma cells obtained are sown at a density of 1×10⁵ cells per ml in RPMI 1640 medium containing 10% FCS and proliferated for 7 days in a fermenter (Thermodux Co., Wertheim/Main, Model MCS-104XL, Order No. 144-050). On average concentrations of 100 μg monoclonal antibody per ml are obtained in the culture supernatant. Purification of this antibody from the culture supernatant is carried out by conventional methods in protein chemistry (e.g. according to Bruck, C., et al., Methods Enzymol. 121 (1986) 587-695).

Generation of Polyclonal Antibodies

a) Immunization

For immunization, a fresh emulsion of the protein solution (100 μg/ml PDX1 or hemocyanin-peptide-conjugate) and complete Freund's adjuvant at the ratio of 1:1 is prepared. Each rabbit is immunized with 1 ml of the emulsion at days 1, 7, 14 and 30, 60 and 90. Blood is drawn and resulting anti-PDX1 serum used for further experiments as described in Examples 3 and 4.

b) Purification of IgG (Immunoglobulin G) from Rabbit Serum by Sequential Precipitation with Caprylic Acid and Ammonium Sulfate

One volume of rabbit serum is diluted with 4 volumes of acetate buffer (60 mM, pH 4.0). The pH is adjusted to 4.5 with 2 M Tris-base. Caprylic acid (25 μl/ml of diluted sample) is added drop-wise under vigorous stirring. After 30 min the sample is centrifuged (13,000×g, 30 min, 4° C.), the pellet discarded and the supernatant collected. The pH of the supernatant is adjusted to 7.5 by the addition of 2 M Tris-base and filtered (0.2 μm).

The immunoglobulin in the supernatant is precipitated under vigorous stirring by the drop-wise addition of a 4 M ammonium sulfate solution to a final concentration of 2 M. The precipitated immunoglobulins are collected by centrifugation (8,000×g, 15 min, 4° C.).

The supernatant is discarded. The pellet is dissolved in 10 mM NaH₂PO₄/NaOH, pH 7.5, 30 mM NaCl and exhaustively dialyzed. The dialysate is centrifuged (13,000×g, 15 min, 4° C.) and filtered (0.2 μm).

Biotinylation of Polyclonal Rabbit IgG

Polyclonal rabbit IgG is brought to 10 mg/ml in 10 mM NaH₂PO₄/NaOH, pH 7.5, 30 mM NaCl. Per ml IgG solution 50 μl Biotin-N-hydroxysuccinimide (3.6 mg/ml in DMSO) are added. After 30 min at room temperature, the sample is chromatographed on Superdex 200 (10 mM NaH₂PO₄/NaOH, pH 7.5, 30 mM NaCl). The fraction containing biotinylated IgG are collected. Monoclonal antibodies are biotinylated according to the same procedure.

Digoxygenylation of Polyclonal Rabbit IgG

Polyclonal rabbit IgG is brought to 10 mg/ml in 10 mM NaH₂PO₄/NaOH, 30 mM NaCl, pH 7.5. Per ml IgG solution 50 μl digoxigenin-3-O-methylcarbonyl-ε-aminocaproic acid-N-hydroxysuccinimide ester (Roche Diagnostics, Mannheim, Germany, Cat. No. 1 333 054) (3.8 mg/ml in DMSO) are added. After 30 min at room temperature, the sample is chromatographed on Superdex® 200 (10 mM NaH₂PO₄/NaOH, pH 7.5, 30 mM NaCl). The fractions containing digoxigenylated IgG are collected. Monoclonal antibodies are labeled with digoxigenin according to the same procedure.

### EXAMPLE 3

### Western Blot for the Detection of PDX1 in Human Serum and Plasma Samples

SDS-PAGE and Western Blotting are carried out using reagents and equipment of Invitrogen, Karlsruhe, Germany. Human plasma samples are diluted 1:20 in reducing NUPAGE (Invitrogen) LDS sample buffer and heated for 5 min at 95° C. 10 μl aliquots are run on 4-12% NuPAGE gels (Bis-Tris) in the MES running buffer system. The gel-separated protein mixture is blotted onto nitrocellulose membranes using the Invitrogen XCell II Blot Module (Invitrogen) and the NuPAGE transfer buffer system. The membranes are washed 3 times in PBS/0.05% TWEEN 20 and blocked with SuperBlock Blocking Buffer (Pierce Biotechnology, Inc., Rockford, Ill., USA). The biotinylated primary antibody is diluted in SuperBlock Blocking Buffer (0.01-0.2 μg/ml) and incubated with the membrane for 1 h. The membranes are washed 3 times in PBS/0.05% TWEEN 20. The specifically bound biotinylated primary antibody is labeled with a streptavidin-HRP-conjugate (20 mUABTS/ml in SuperBlock Blocking Buffer). After incubation for 1 h, the membranes are washed 3 times in PBS/0.05% TWEEN 20. The bound streptavidin-HRP-conjugate is detected using a chemiluminescent substrate (SuperSignal West Femto Substrate, Pierce Biotechnology, Inc., Rockford, Ill., USA) and autoradiographic film. Exposure times varies from 10 min to over night.

### EXAMPLE 4

### ELISA for the Measurement of PDX1 in Human Serum and Plasma Samples

For detection of PDX1 in human serum or plasma, a sandwich ELISA is developed. For capture and detection of the antigen, aliquots of the anti-PDX1 polyclonal antibody (see Example 2) are conjugated with biotin and digoxygenin, respectively.

Streptavidin-coated 96-well microwell plates are incubated with 100 μl biotinylated anti-PDX1 polyclonal antibody for 60 min at 10 μg/ml in 10 mM phosphate, pH 7.4, 1% BSA, 0.9% NaCl and 0.1% TWEEN 20. After incubation, plates are washed three times with 0.9% NaCl, 0.1% TWEEN 20. Wells are then incubated for 2 h with either a serial dilution of the recombinant protein (see Example 2) as standard antigen or with diluted plasma samples from patients. After binding of PDX1, plates are washed three times with 0.9% NaCl, 0.1% TWEEN 20. For specific detection of bound PDX1, wells are incubated with 100 μl of digoxygenylated anti-PDX1 polyclonal antibody for 60 min at 10 μg/ml in 10 mM phosphate, pH 7.4, 1% BSA, 0.9% NaCl and 0.1% TWEEN 20. Thereafter, plates are washed three times to remove unbound antibody. In a next step, wells are incubated with 20 mU/ml anti-digoxigenin-POD conjugates (Roche Diagnostics GmbH, Mannheim, Germany, Catalog No. 1633716) for 60 min in 10 mM phosphate, pH 7.4, 1% BSA, 0.9% NaCl and 0.1% TWEEN 20. Plates are subsequently washed three times with the same buffer. For detection of antigen-antibody complexes, wells are incubated with 100 μl ABTS solution (Roche Diagnostics GmbH, Mannheim, Germany, Catalog No. 11685767) and OD is measured after 30-60 min at 405 nm with an ELISA reader.

### EXAMPLE 5

### ROC Analysis to Assess Clinical Utility in Terms of Diagnostic Accuracy

Accuracy is assessed by analyzing individual liquid samples obtained from well-characterized patient cohorts, i.e., 50 patients having undergone mammography and found to be free of BC, 50 patients each diagnosed and staged as invasive ductal and invasive lobular T1-3, N0, M0 of BC, 50 patients diagnosed with progressed BC, having at least tumor infiltration in at least one proximal lymph node or more severe forms of metastasis, 50 patients each diagnosed with medullary, mucinous, tubular, or papillary breast carcinoma, and 50 patients diagnosed with DCIS, respectively. CA 15-3 as measured by a commercially available assay (Roche Diagnostics, CA 15-3-assay (Cat. No. 0 304 5838 for ELECSYS Systems immunoassay analyzer) and PDX1 measured as described above have been quantified in a serum obtained from each of these individuals. ROC-analysis is performed according to Zweig, M. H., and Campbell, supra. Discriminatory power for differentiating patients in the group Tᵢₛ-3, N0, M0 from healthy individuals for the combination of PDX1 with the established marker CA 15-3 is calculated by regularized discriminant analysis (Friedman, J. H., Regularized Discriminant Analysis, Journal of the American Statistical Association 84 (1989) 165-175).

Preliminary data indicate that PDX1 may also be very helpful in the follow-up of patients after surgery.

What is claimed is:

1. A method for assessing breast cancer in a patient comprising:
measuring in a sample from said patient a concentration of peroxiredoxin 1 (PDX1), and
using the concentration in the assessment of the presence of breast cancer.

2. The method of claim 1 wherein said sample is serum.

3. The method of claim 1 wherein said sample is plasma.

4. The method of claim 1 wherein said sample is whole blood.

5. The method of claim 1 wherein said sample is nipple aspirate fluid.

6. The method of claim 1 wherein the patient is a breast cancer patient in stage Tᵢₛ-3; N0; M0.

7. The method of claim 1 further comprising the step of measuring in said sample a concentration of a known marker of breast cancer and including the concentration of the known marker in the assessment of breast cancer.

8. The method of claim 7 wherein said known marker is selected from the group consisting of carcinoembryonic antigen (CEA), cancer antigen 15-3 (CA 15-3), cellular retinoic acid-binding protein II (CRABP-II), and apoptosis-associated speck-like protein containing a caspase-associated recruitment domain (ASC).

9. The method of claim 8 wherein said known marker is CEA.

10. The method of claim 8 wherein said known marker is CA 15-3.

11. The method of claim 8 wherein said known marker is CRABP-II.

12. The method of claim 8 wherein said known marker is ASC.

13. A marker panel comprising a specific binding agent for PDX1 and a specific binding agent for a known marker of breast cancer.

14. The marker panel of claim 13 wherein said known marker is selected from the group consisting of carcinoembryonic antigen (CEA), cancer antigen 15-3 (CA 15-3), cellular retinoic acid-binding protein II (CRABP-II), and apoptosis-associated speck-like protein containing a caspase-associated recruitment domain (ASC).

15. A kit for assessing breast cancer in a patient, said kit comprising reagents for measuring PDX1.

16. The kit of claim 15 further comprising reagents for measuring a known marker of breast cancer selected from the group consisting of carcinoembryonic antigen (CEA), cancer antigen 15-3 (CA 15-3), cellular retinoic acid-binding protein II (CRABP-II), and apoptosis-associated speck-like protein containing a caspase-associated recruitment domain (ASC).


SEQUENCE LISTING (8 sequences)

SEQ ID NO: 1 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 2 - protein, 8 aa, Homo sapiens
GLFIIDDK

SEQ ID NO: 3 - protein, 12 aa, Homo sapiens
GLFIIDDKGILR

SEQ ID NO: 4 - protein, 25 aa, Homo sapiens
KLNCQVIGASVDSHFCHLAWVNTPK

SEQ ID NO: 5 - protein, 17 aa, Homo sapiens
KQGGLGPMNIPLVSDPK

SEQ ID NO: 6 - protein, 24 aa, Homo sapiens
LNCQVIGASVDSHFCHLAWVNTPK

SEQ ID NO: 7 - protein, 10 aa, Homo sapiens
LVQAFQFTDK

SEQ ID NO: 8 - protein, 18 aa, Homo sapiens
TIAQDYGVLKADEGISFR